(19) United States
(12) Patent Application Publication
(10) Pub. No.: US 2011/0276080 A1
(43) Pub. Date: Nov. 10, 2011

(54) INTRAVASCULAR DEVICE, METHOD FOR MANUFACTURING SAME, AND KITS INCLUDING SAME

(75) Inventor: Alain Nigon, Bormes les Mimosas (FR)

(73) Assignee: A.L.N., Ghisonaccia (FR)

(21) Appl. No.: 13/145,403

(22) PCT Filed: Jan. 27, 2010
(86) PCT No.: PCT/FR2010/050123

§ 371 Date: Jul. 20, 2011

Related U.S. Application Data
(60) Provisional application No. 61/153,031, filed on Feb. 17, 2009.

(30) Foreign Application Priority Data
Feb. 3, 2009 (FR) 0950675

(51) Int. Cl. A61M 29/00; B23P 11/00

(52) U.S. Cl. 606/194; 29/428

(57) ABSTRACT

The invention relates to an intravascular device (1) that can be used in pairs or more for filling a same aneurysm, essentially consisting of a skeleton covering with a film for forming a pouch that can be filled with blood, said skeleton being made of jointed resilient strands (2,3,4,5,6) forming, in the absence of stress, an elongate, partially planar pouch tapering into a tip at the ends (7,8) thereof, and having, when applied with stress, an average general diameter enabling the insertion thereof into a catheter having a 24F size (8 mm diameter) or less, said pouch being provided with an opening letting blood into said pouch. The invention also relates to a method for manufacturing same and kits containing same.

The present invention relates to an intravascular device, to its method of manufacture and to kits containing it.

An aneurism is a permanent and localized distension of an artery, representing an at least 50% increase in its diameter in relation to the normal figures for the artery in question (as defined by the North American Society for Vascular Surgery).

In practice, the abdominal aorta is said to display an aneurism if the diameter of the aorta is greater than 35 mm for men and 30 mm for women.

When the aneurism reaches 5 cm, the patient is hospitalized so that the aneurism sac can be isolated from the arterial blood circulation. The purpose of the intervention is to introduce an aorta prosthesis using an endovascular route. This prosthesis is aimed at excluding the aneurism from the blood pressure and thus at preventing it from spreading.

At the present time, there are two therapeutic methods that can be offered to patients.

The conventional surgical method involves opening up the abdomen and fitting a prosthesis made of an impervious fabric. The results of this type of treatment are well known, but the treatment does have the disadvantage of requiring surgery and blood transfusions and a lengthy hospital stay and lengthy convalescence period.

The method using the endovascular route carries out the same type of treatment but without the surgical intervention. In this case, an endoprosthesis consisting of a metal spring covered with an impervious fabric is introduced via a femoral artery in the groin and is then deployed in the aorta to exclude this aneurism. This recent method, although performed under general anaesthetic, appears to be less aggressive than a surgical bypass but its long-term outcome is not yet fully known.

Whatever the therapeutic method used, it is based on excluding the aneurism from aorta blood pressure, making it possible to prevent this aneurism from extending. Further, this endoprosthesis makes it possible to avoid the risk of the clots that develop in the aneurism migrating.

An endoprosthesis consists of a metal part covered with a coating able to act as a barrier against the blood, made up of biocompatible materials intended for medical use. This endoprosthesis is compressed in an installation system comprising a carrying catheter. It is then introduced using a metal guide and a catheter as far as the implantation site. At the lesion, this endoprosthesis is released into the lumen of the aorta by withdrawal of the installation system. It then spontaneously attaches itself to the wall of the aorta.

The literature describes a significant rate of complications with this type of intervention. These complications notably include complications known as “endoleaks”, which occur at the point of attachment of the endoprosthesis or through the endoprosthesis itself, and limit the aneurism exclusion effectiveness. They also occur through blood arriving directly into the aneurism sac via collateral arteries such as the mesenteric arteries. The blood thus continues to flow into the aneurism sac and the aneurism grows and then finally ruptures (proving fatal in 98% of cases).

It would therefore be particularly desirable to reduce or eliminate these complications.

Now, following lengthy research into new endoprostheses, the applicant has set off in an entirely different direction and has developed a device to be fitted, not in place of, but together with, an aortic endoprosthesis.

Briefly, the new device is, in general terms, a mechanical device that fills the space between the endoprosthesis and the arterial wall of the aneurism sac.

This is why a subject of the present application is, more specifically, an intravascular device for use in pairs or more to fill one and the same aneurism, the device being essentially made up of a skeleton covered with a film to form a pouch that can become filled with blood, said skeleton being formed of jointed resilient strands which, when not under stress, form a partially flattened elongate pouch that tapers to a point at its ends and when under stress having a mean overall diameter that will allow it to be introduced through a 24 F (8 mm diameter) or smaller catheter, said pouch being equipped with an orifice to allow blood to enter said pouch.

The overall structure of the intravascular device of the invention is of the type of a skeleton defining the volume of the device in the three dimensions, covered by a flexible covering.

Note that in the present application, conventionally the indefinite article “a/an/one” is to be considered as a generic plural (meaning “at least a/an/one” or else “one or more”), except when the context indicates the contrary (1 or “a/one single”). Thus, for example, when it is stated hereinafter that an intravascular device is installed, this means that one or more intravascular devices are installed.

Under preferred conditions for the implementation of the invention, in section transverse to its major axis, an intravascular device of the invention has the overall shape of a semicircle or of a third of a circle.

Under other preferential conditions for the implementation of the invention, the mechanical skeleton is made up of wires or cables.

A bioimplantable material is used to produce these wires or cables. These wires or cables may for example be made of a plastic such as polyethylene or polypropylene, and particularly of a metal or alloy such as stainless steel, preferably a non-magnetic stainless steel A316LVM, or alternatively phinox, nitinol alloy NiTi, or titanium.

The metals/alloys or any other material used for the mechanical structure or the covering or both may then be treated with a polymer or a carbon coating, for example applied by plasma deposition, in order to modify the surface properties thereof. Thus, for example, it is possible to reduce the risks of infection, induce a hydrophobic or alternatively hydrophilic effect.

The wires or cables are shaped with a view to the deployed (unstressed) final shape. They may notably be crimped and/or welded together at the ends of the intravascular device of the invention. The ends may, for example, be of cylindrical, ovoid or other shape, preferably of a non-traumatic shape (neither pointed nor cutting). They are preferably cylindrical ending in a hemisphere. They may be fitted with an annular hollow. The strands may also be fixed at each end to a semi-annular connecting element so that they better match the shape of an endoprosthesis. The semi-annular connecting element is then flexible so that it can be introduced into an introducing catheter of appropriate diameter.

The diameter of these wires or cables can adopt various values according to the nature of the material used. When made of stainless steel, the diameter of these wires may range from 0.2 to 0.5, preferably from 0.3 to 0.4 mm, and in particular may have a value of approximately 0.3 mm.

The number of these wires or cables may notably range from 4 to 12, preferably from 4 to 10, notably from 5 to 10, and more particularly from 5 to 8.

The length of an intravascular device of the invention when unstressed is advantageously from 6 to 12, preferably from 7 to 11, more particularly from 8 to 10 cm.

The maximum width of a device is advantageously from 3 to 8, preferably from 2 to 8, notably from 2 to 6, more particularly from 5 to 6 cm.

The maximum height of a device (measured perpendicular to the flattened region) is advantageously from 1.0 to 4.5, preferably from 1.0 to 4, notably from 1.2 to 3, more particularly around 3 cm.

Devices with a length from 7 to 11 cm, a maximum width from 2 to 6 cm and a maximum height from 0.8 to 4 cm are notably preferred.

As has been seen, the skeleton is covered with a film so that a pouch can be formed in an aneurism sac.

The skeleton is covered with a covering, notably a woven or knitted fabric, or even for preference with a polymer film, that adheres to the mechanical structure.

This covering may be impervious to blood or permeable or semi-permeable to blood. The blood coagulates on the cover and inside the pouch.

There are various materials that can be used including: polyester polyurethane, nylon, dacron, PET or a combination of these materials with other polymer fibers.

The polymer film can be made of polyurethane for example or from any other biocompatible material that can implanted in man, notably a silicone film.

An orifice to allow blood to enter said pouch has, for example, a surface area corresponding to that of a circle with a diameter of 0.5 to 55 mm, preferably 0.5 to 30 mm, advantageously 0.5 to 3.5, preferably 0.5 to 3, notably 1 to 2.5 mm. An orifice that has a surface area corresponding to that of a circle of a diameter of 10 to 30 mm is particularly preferred.

An orifice to allow blood to enter said pouch may just as well be provided in the partially flattened part of the device as in the bulging part notably having the overall shape of a semicircle or a third of a circle in section transverse to its major axis. Its shape may be any, just as well circular as in the shape of a barrel or barrel section, or alternatively square or rectangular.

Under still other preferred conditions of implementation of the invention, radiopaque markers are provided on the wires or cables of the mechanical skeleton, or alternatively on the covering. These markers are advantageously arranged in such a way that they become superposed on one another when the device 1 according to the invention is viewed from the side. It is thus possible to determine the position of the device with respect to its axis of rotation.

Another subject of the present application is a method of manufacturing an abovementioned intravascular device, characterized in that a skeleton is created that is formed of resilient strands shaped by grouping the strands together at their ends, giving them the desired three-dimensional shape, and then securing the ends together to allow them to maintain the desired three-dimensional shape and covering this skeleton with a film pierced with an orifice.

These wires or cables may be bonded together. Under preferred conditions of implementation of the abovementioned method, they are crimped and/or welded together.

Under other preferred conditions of implementation of the abovementioned method, the film that is impervious to blood is bonded onto the wires or cables or applied thereto using a thermal process.

The intravascular devices that form the subject of the present invention have very attractive properties. They are able to alleviate the problems encountered following the placement of an aortic endoprosthesis in patients suffering, for example, from an Abdominal Aortic Aneurism (AAA). They encourage the blood to coagulate and mechanically hold the endoprosthesis in place. This coagulation of the blood is improved through a suitable choice of the nature of the film.

They are able to support the endoprosthesis and restrict its possibilities of movement within the aneurism sac. In addition, they allow the blood present in the aneurism sac to coagulate. A high degree of thrombosis (formation of clots) indeed limits leaks in the aneurism sac. This technique has the advantage over polymer adhesives notably in that it mechanically supports the endoprosthesis.

These properties are illustrated hereinafter in the experimental part. They justify the use of the abovedescribed intravascular devices in a method, notably a preventive method, of treating the complications, particularly those termed “endoleaks”, associated with the fitting of an endoprosthesis in a blood vessel, notably a artery such as the aorta.

This is why a further subject of the present invention is a method, notably a preventive method, of treating the complications, particularly those termed “endoleaks”, associated with the fitting of an endoprosthesis in a blood vessel, notably an artery such as the aorta, characterized in that two or more abovedescribed intravascular devices are installed in the aneurism sac in order to fill it.

The intravascular devices according to the invention can be used as follows, in general. Two or more abovedescribed intravascular devices are implanted in the aneurism sac via an endovascular route following the implantation of an endoprosthesis or at the same time thereas. They are brought into position using a catheter and deploy between the aortic endoprosthesis and the dilated wall of the physiological blood vessel.

They may notably be used as follows, in the case of an intravascular device of the invention with the overall shape of a semicircle in section transverse to its major axis and implanted in the aorta. The general principle remains the same for other vessels.

Use will be made of two intravascular devices which will be implanted at the same time as the endoprosthesis or after the latter.

For each of the three items to be installed (the endoprosthesis and the intravascular devices of the invention), the following equipment is used for implantation:
a puncture needle to provide access to the femoral artery,
a catheter/dilator set to carry the equipment,
a pushing catheter to push the equipment.

In order to install or check the position of the device according to the invention in the aneurism sac, radiopaque markers are preferably provided on the wires or cables of the mechanical structure or else on the covering. These two (or more) markers are positioned in such a way that they become superposed when the device 1 according to the invention is viewed from the side. It is thus possible to determine the position of the device with respect to its axis of rotation.

Likewise, one or more markers is or are advantageously provided on the catheters or dilators, or on both.

Use is also made of a J-shaped guide (for example a 0.018 inch=0.46 mm guide) and of a 5 F or 4 F catheter for each intravascular device of the invention.

Also for each intravascular device of the invention use is made of a J-shaped guide (for example a 0.035 inch=0.89 mm guide) to reach as far as the implantation site.

The main steps involved in implanting an aortic endoprosthesis are as follows:

A puncture is made in the groin and a J-shaped metal guide is inserted as far as the implantation site. The guide acts as a support to guide the catheter in which the endoprosthesis is inserted.

The endoprosthesis is released at the aneurism.

When the endoprosthesis is fully deployed, the aneurism sac is isolated from the blood circulation.

The same procedure is followed with the first and then the second abovedescribed intravascular device. When the catheter is withdrawn in order thus to release the device of the invention at the site of the aneurism, the device deploys of its own accord in the aneurism sac because of the resilience of the material of which the skeleton is made. The covering fixed to the skeleton structure deploys at the same time as the skeleton structure and therefore the volume thus created occupies the empty space of the aneurism sac.

One subject of the present application is thus a method for occluding an aneurism pouch in which
a puncture is made,
a J-shaped metal guide is inserted as far as the implantation site,
a catheter/dilator set is guided as far as the aneurism sac,
the J-shaped guide and the dilator are withdrawn. The catheter remains in position in the aneurism sac,
an abovedescribed intravascular device is inserted into the catheter, then pushed forward as far as the end of said catheter using a pusher,
the intravascular device is held in place using this pusher,
said catheter is withdrawn in order thus to release the device into the aneurism pouch,
said pusher and the supporting catheter are withdrawn from the patient,
optionally, one or more other above-mentioned intravascular devices is/are installed.

Conventionally, hemostasis is then performed at the site of the puncture.

Another subject of the present application is a kit (or set) comprising
an abovedescribed intravascular device, and
a catheter/dilator set capable of carrying an intravascular device of the invention.

Another subject of this invention is a kit comprising
two or three abovedescribed intravascular devices,
two or three catheter/dilator sets each carrying an intravascular device of the invention as far as the implantation site.

The abovementioned kits preferably further comprise one or both of the following components:
one or more J-shaped guides for reaching as far as the implantation site,
a pushing catheter for pushing an intravascular device of the invention.

Equally a subject of the invention is one of the above kits, additionally containing
an endoprosthesis,
a catheter/dilator set that can be used to introduce and install the endoprosthesis,
a pushing catheter for pushing the endoprosthesis.

Another subject of the invention is one of the above kits, further containing instructions for use of the equipment in the kit.

Yet another subject of the invention is one of the above kits, further containing a needle for puncturing.

It is preferable for the intravascular devices of the invention and the endoprosthesis to be installed during the same intervention.

The abovedescribed preferred conditions for implementation of the intravascular devices according to the invention also apply to the other subjects of the invention mentioned hereinabove, notably to the methods of manufacturing them, to the kits and to the methods of use.

The invention will be better understood if reference is made to the attached drawings in which:

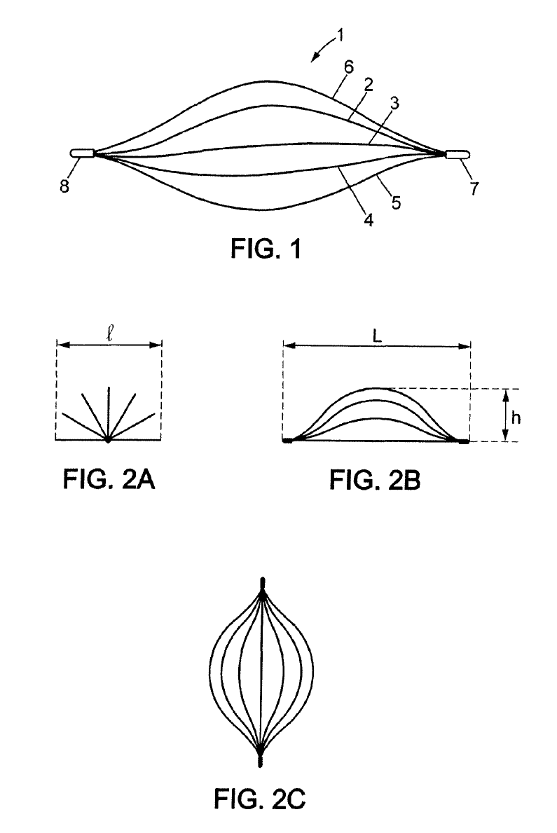

FIG. 1 depicts a view from above of an intravascular device according to the invention;

FIG. 2 depicts a diagram of the skeleton of an intravascular device according to the invention, viewed end-on along its major axis (2A), from the side (2B), and from above (2C);

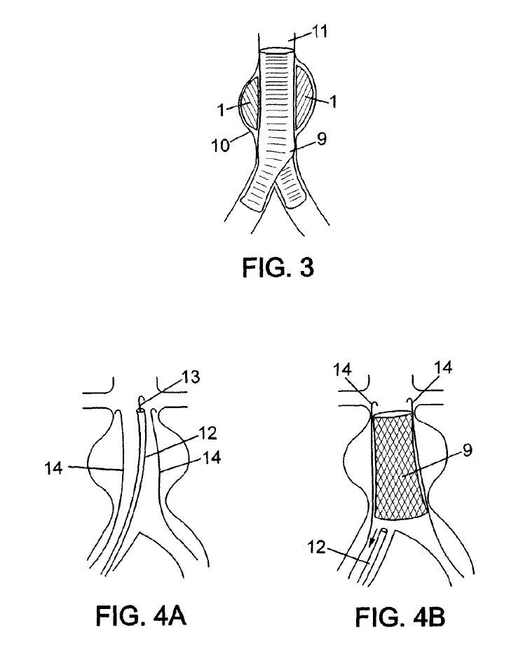

FIG. 3 is a schematic depiction of the installation of devices according to the invention in such a way as to fill the aneurism sac, the devices according to the invention being depicted in section;

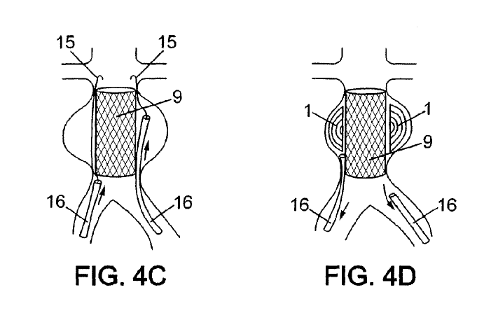

FIG. 4 is a schematic depiction of the installation of a device of the invention.

FIG. 1 shows, lying on its flattened face, an intravascular device 1 according to the invention.

It comprises a skeleton made up of seven bent metal wires of which only five 2, 3, 4, 5, 6 are visible in the figure, two wires being hidden by two other wires. The left 7 and right 8 ends of these wires have been brought together and crimped together. The left 7 and right 8 ends of the device are rounded so as to be non-traumatic in order to avoid them creating lesions in the vascular wall. They are cylindrical, ending in a hemisphere.

Geometrically, in space, the overall shape of the device would be that of a rugby ball cut along its middle along its longest length, and with its ends elongated to a point.

The skeleton is completely covered with a film (not depicted), in this instance made of polyurethane. An orifice to allow blood to enter said pouch is not depicted either.

FIGS. 2A, 2B and 2C illustrate the geometry of the device of the invention and give an understanding of its dimensions.

Various embodiments have been produced. In these, the length (L) of the device adopted various values ranging from 6 to 12 cm.

The height h adopted values ranging from 1.5 to 4.5 cm, and the width 1 values from 3 to 8 cm.

In the various embodiments, the dimensions were approximately homothetic.

FIG. 3 is a diagrammatic depiction of the devices 1 according to the invention installed around an endoprosthesis 9 in such a way as to almost entirely mechanically fill the aneurism sac 10 of an aorta 11.

FIG. 4 illustrates a procedure for installing devices according to the invention in such a way as to fill the aneurism sac.

Use is made of the following equipment:
three puncture needles,
two J-shaped guides 14 with a diameter of 0.018 inches,
a complete endoprosthesis introduction system,
an endoprosthesis,
two catheters with an inside diameter 5 F,
two dilators with an outside diameter 4.5 F,
three J-shaped guides 13, 15 with a diameter of 0.035 inches,
a catheter 16 (inside diameter ID 10 F—3.3 mm in diameter—to guide a 1st device of the invention) and a catheter 16 (inside diameter ID 10 F, to guide a second device of the invention),
two dilators with OD (OD=outside diameter) 9.5 F to guide the 10 F catheters,
two devices 1 according to the invention,
two 9.5 F OD pushing catheters of diameter 8.

Two devices 1 according to the invention are collapsed by using the elasticity of the skeleton and of the film to place them under stress so that they can be introduced into catheters of inside diameter 10 F.

The conventional steps of this type of intervention such as disinfection, creation of sterile fields, anesthesia or rinsing will not be described.

The procedure is as follows:
puncturing the right and left femoral arteries,
inserting the flexible end of the 0.018 J-guides 14 into the needles and routing them in order to position the end of the 0.018 J-guide 14 in the aneurism sac via the right and left femoral arteries,
holding the 0.018 J-guides 14 in place and withdrawing the needle intended for the endoprosthesis guide,
inserting the flexible end of the 0.035 J-guide 13 in a third needle,
routing it to position the end of the 0.035 J-guide 13 in the aneurism sac,
holding the 0.035 J-guide 13 in place and withdrawing the needle,
progressing the endoprosthesis 9 introduction system 12 along the 0.035 J-guide 13 (FIG. 4A) and deploying the endoprosthesis 9 at the aneurism 10 keeping the 0.018 J-guides 14 between the endoprosthesis 9 and the wall of the artery (FIG. 4B).

Hereinbelow, the procedures are carried out via the left femoral artery and via the right femoral artery:
inserting the outer part of the 0.018 J-guide 14 in the distal end of the catheter/dilator set,
routing the catheter/dilator set along the artery by sliding it along the 0.018 J-guide 14,
introducing the catheter/dilator set between the endoprosthesis 9 and the arterial wall and routing the end of the set into the aneurism sac,
withdrawing the 0.018 J-guide 14 while holding the catheter/dilator set in position,
introducing the 0.035 J-guide 15 into the catheter/dilator set,
withdrawing the catheter/dilator set leaving the 0.035 J-guide 15 in the aneurism sac,
routing the outer part of the J-guide through the distal end of the introduction system 16,
routing the introduction system 16 (FIG. 4C) of the device according to the invention (10 F ID catheter+9.5 F OD dilator) along the artery by sliding it along the 0.035 J-guide,
routing the end of the introduction system 16 and inserting it between the endoprosthesis and the arterial wall,
withdrawing the J-guide 15 and the dilator from the 9.5 F OD introduction system 16,
introducing the device 1 according to the invention into the 10 F ID introduction catheter 16,
routing the device 1 according to the invention as far as the distal end of the 10 F ID catheter 16, using the pushing catheter,
sliding the 10 F introduction catheter 16 along the pushing catheter with a slow withdrawal movement until the device 1 according to the invention is released,
withdrawing the introduction catheter 16 and the pushing catheter (FIG. 4D).

1. An intravascular device (1) for use in pairs or more to fill one and the same aneurism, the device being essentially made up of a skeleton covered with a film to form a pouch that can become filled with blood, said skeleton being formed of jointed resilient strands which, when not under stress, form a partially flattened elongate pouch that tapers to a point at its ends and when under stress having a mean overall diameter that will allow it to be introduced through a 24 F (8 mm diameter) or smaller catheter, said pouch being equipped with an orifice to allow blood to enter said pouch.

2. The intravascular device as claimed in claim 1, characterized in that, in section transverse to its major axis, it has the overall shape of a semicircle or a third of a circle.

3. The intravascular device as claimed in claim 1, characterized in that the number of strands is from 4 to 10.

4. The intravascular device as claimed in claim 1, characterized in that the orifice for allowing blood to enter has a surface area corresponding to that of a circle with a diameter of 0.5 to 55 mm.

5. The intravascular device as claimed in claim 1, characterized in that, when not stressed, it has a length from 7 to 11 cm.

6. The intravascular device as claimed in claim 1, characterized in that the skeleton is covered with a polymer film adhering to the skeleton.

7. The intravascular device as claimed in claim 1, characterized in that it has a length from 7 to 11 cm, a maximum width from 2 to 6 cm, and a maximum height from 1.0 to 4 cm.

8. A method of manufacturing an intravascular device as defined in claim 1, characterized in that a skeleton is created that is formed of resilient strands shaped by grouping the strands together at their ends, giving them the desired three-dimensional shape, and then securing the ends together to allow them to maintain the desired three-dimensional shape and covering this skeleton with a film that is impervious to blood in order to form a pouch that is impervious to blood.

9. A kit comprising
an intravascular device as defined in claim 1, and
a catheter/dilator set capable of carrying one aforesaid intravascular device.

10. The kit as claimed in claim 9, characterized in that it further comprises one or both of the following components:
one or more J-shaped guides for reaching as far as the implantation site,
a pushing catheter for pushing an intravascular device of the invention.
